(19) United States
(12) Patent Application Publication
(10) Pub. No.: US 2017/0051249 A1
(43) Pub. Date: Feb. 23, 2017

(54) MANIPULATION OF OVARIAN PRIMORDIAL FOLLICLES

(71) Applicant: The Board of Trustees of the Leland Stanford junior University, Stanford, CA (US)

(72) Inventors: Aaron J.W. Hsueh, Stanford, CA (US); Jing Li, Nanjing (CN)

(21) Appl. No.: 15/274,949

(22) Filed: Sep. 23, 2016

Related U.S. Application Data
(63) Continuation of application No. 12/657,679, filed on Jan. 25, 2010, now abandoned.
(60) Provisional application No. 61/206,131, filed on Jan. 27, 2009.
(60) Provisional application No. 61/205,771, filed on Jan. 23, 2009.

(51) Int. Cl. C12N 5/075; A61K 38/24; A61K 35/54; A61K 31/555

(52) U.S. Cl. CPC C12N 5/0609; A61K 31/555; A61K 38/24; A61K 35/54; C12N 2501/31; C12N 2501/999; C12N 2501/998; A61B 17/435

(57) ABSTRACT

Methods are provided for activating dormant ovarian primordial follicles in a mammal to promote development to preovulatory follicles.

### CROSS REFERENCE

This application claims benefit and is a Continuation of application of Ser. No. 12/657,679 filed Jan. 25, 2010, which claims benefit of U.S. Provisional Patent Application No. 61/206,131, filed Jan. 27, 2009 and U.S. Provisional Patent Application No. 61/205,771, filed Jan. 23, 2009, which applications are incorporated herein by reference in their entirety.

### FEDERALLY SPONSORED RESEARCH AND DEVELOPMENT

This invention was made with Government support under grant HD060864 awarded by the National Institutes of Health. The Government has certain rights in this invention.

### BACKGROUND OF THE INVENTION

The growth and maturation of mammalian germ cells is intricately controlled by hormones; including gonadotropins secreted by the anterior pituitary; and local paracrine factors. The majority of the oocytes within the adult human ovary are maintained in prolonged stage of first meiotic prophase; enveloped by surrounding follicular somatic cells. Periodically, a group of primordial follicles enters a stage of follicular growth. During this time, the oocyte undergoes a large increase in volume, and the number of follicular granulosa cells increase. The maturing oocyte synthesizes paracrine factors that allow the follicle cells to proliferate, and the follicle cells secrete growth and differentiation factors (for example TGF-β2, VEGF, leptin, and FGF2) that enhance angiogenesis and allow the oocyte to grow. After progressing to a certain stage, oocytes and their follicles die, unless they are exposed to gonadotropic hormones that prevent somatic cell apoptosis and induce meiotic maturation in the oocyte.

Mammalian ovaries consist of follicles as basic functional units. The total number of ovarian follicles is determined early in life, and the depletion of this pool leads to reproductive senescence. Each follicle develops to either ovulate or, more likely, to undergo degeneration. Individual follicles consist of an innermost oocyte, surrounding granulosa cells, and outer layers of thecal cells. The fate of each follicle is controlled by endocrine as well as paracrine factors. The follicles develop through primordial, primary, and secondary stages before acquiring an antral cavity. At the antral stage a few follicles, under the cyclic gonadotropin stimulation that occurs after puberty, reach the preovulatory stage and become a major source of the cyclic secretion of ovarian estrogens in women of reproductive age. In response to preovulatory gonadotropin surges during each reproductive cycle, the dominant Graafian follicle ovulates to release the mature oocyte for fertilization, whereas the remaining theca and granulosa cells undergo transformation to become the corpus luteum.

Throughout the reproductive life, primordial follicles undergo initial recruitment to enter the growing pool of primary follicles. In the human ovary, greater than 120 days are required for the primary follicles to reach the secondary follicle stage, whereas 71 days are needed to grow from the secondary to the early antral stage. Once initiated to enter the growing pool, ovarian follicles progress to reach the antral stage and minimal follicle loss was found until the early antral stage. During cyclic recruitment, increases in circulating FSH allow a cohort of antral follicles to escape apoptotic demise. Among this cohort, a leading follicle emerges as dominant by secreting high levels of estrogens and inhibins to suppress pituitary FSH release. The result is a negative selection of the remaining cohort, leading to its ultimate demise. Concomitantly, increases in local growth factors and vasculature allow a positive selection of the dominant follicle, thus ensuring its final growth and eventual ovulation and luteinization. After cyclic recruitment, it takes only 2 weeks for an antral follicle to become a dominant Graafian follicle. The overall development of human follicles from primordial to preovulatory stages require more than six months.

In rodents, the duration of follicle development is much shorter than that needed for human follicles. The time required between the initial recruitment of a primordial follicle and its growth to the secondary stage is more than 30 days, whereas the time for a secondary follicle to reach the early antral stage is about 28 days. Once reaching the early antral stage, the follicles are subjected to cyclic recruitment, and only 2-3 days are needed for them to grow into preovulatory follicles.

The development of follicles from the smallest primordial and primary follicles to the largest preovulatory follicles requires different stage-dependent stimulatory and survival factors. FSH, activin, Nerve growth factor, and GDF-9 are important for the growth and differentiation of primary and/or secondary follicles. The growth of antral and preovulatory follicles is dependent on gonadotropin stimulation and FSH is the major survival factor to rescue early antral follicles from apoptotic demise during cyclic recruitment. Treatment with FSHctp (a long-acting FSH agonist) has resulted in increased ovarian weight and follicle development. Thus, the development of follicles can be divided into gonadotropin-dependent and gonadotropin-responsive stages.

The mechanisms that regulate the gradual exit of ovarian follicles from the non-growing, primordial pool are poorly understood. Whether an individual follicle remains dormant or initiates growth may depend on the balance of stimulatory and inhibitory factors. In a serum-free medium, while bovine primordial follicles become spontaneously activated between 12 and 24 h of culture, only a fraction of human primordial follicles initiate growth in culture. However, this ‘spontaneous’ activation is different from the gradual exit of dormant primordial follicles from the resting pool that occurs in vivo and suggests that primordial follicles in vivo may be subject to a tonic inhibition of growth initiation.

Methods of efficiently activating dormant ovarian primordial follicles is of great interest, including methods for in vitro follicle maturation. The present invention addresses this issue.

### SUMMARY OF THE INVENTION

Methods are provided for activating dormant ovarian primordial follicles in a mammal to promote development to preovulatory follicles. In the methods of the invention, ovarian follicles are transiently exposed in vitro or in vivo to an inhibitor of PTEN (tumor suppressor phosphatase with TENsin homology) at a concentration that is effective to induce the follicles to initiate growth, i.e. to break dormancy. Optionally the follicles are also contacted with a stimulator of PI3K activity. Exposure is normally for a period of from at least about one hour to not more than about 36 hours. Following exposure to a PTEN inhibitor and/or PI3K activator, the follicle may be exposed to follicle stimulating hormone (FSH) or FSH analogs to provide for gonadotropin stimulation. Stimulation with FSH or FSH analogs may be performed in vivo or in vitro, and includes the use of recombinant FSH, naturally occurring FSH in an in vivo host animal, FSH analogs, e.g. FSH-CTP, pegylated forms, and the like.

In some embodiments of the invention, the exposure to a PTEN inhibitor and/or PI3K activator is performed in vitro, e.g. in an organ or tissue culture, where at least one primordial ovarian follicle is transiently exposed to an effective dose of a PTEN inhibitor and/or PI3K activator. The treated follicle may be utilized in vitro, for example for in vitro fertilization, generation of embryonic stem cells, etc., or may be transplanted to provide for in vivo uses. Transplantation modes of interest include, without limitation, transplantation of one or more follicles, including all or a fraction of an ovary, to a kidney capsule, to a subcutaneous site, to an ovarian site, e.g. where one ovary has been retained and one has been removed for ex vivo treatment, the one or more treated follicles may be transplated to the site of the remaining ovary.

In other embodiments, the exposure to a PTEN inhibitor and/or PI3K activator is performed in vivo, where the PTEN inhibitor and/or PI3K activator may be locally or systemically administered to an individual. Following such exposure, the individual may be treated with FSH or FSH analogs, at a concentration that is effective to release a mature oocyte.

In addition to the afore-mentioned PTEN inhibitor and/or PI3K activator and FSH or FSH analogs, the follicles may be exposed in vivo or in vitro to one or more of c-kit ligand, neurotrophins, vascular endothelial growth factor (VEGF), bone morphogenetic protein (BMP)-4, BMP7, leukemia inhibitory factor, basic FGF, keratinocyte growth factor; and the like.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Strong PTEN staining was observed in cumulus and granulosa cells of preovulatory and antral follicles (arrows) whereas the luteal cells were weakly stained (arrowhead).

FIG. 2. PTEN staining was detectable in oocytes of some primordial follicles (solid arrowheads). However, PTEN staining was weaker for other primordial or primary follicles (unfilled arrowheads).

FIG. 3. Control ovaries showed some follicles progressed to preantral follicle (PAF) and antral follicle (AF) stages but with many primordial follicles (white arrowheads) clustered near the cortex region. Follicles entering the growing pool were located in the medulla region.

FIG. 4. In PTEN inhibitor-treated ovaries, most follicles entered the growing pool as reflected by the presence of many preantral follicles (PAF), antral follicles (AF) and a few preovulatory follicles (POF).

FIG. 5. Under a higher magnification, clusters of primordial follicles were evident in the control group (left panel, white arrowheads) but few primordial follicles are present in ovaries treated with the PTEN inhibitor (right panel).

FIG. 6. Treatment with the PTEN inhibitor decreased by 60% the percentage of primordial (Pmd) follicles as compared to controls.

FIG. 7. (upper left), in ovaries incubated with the PTEN inhibitor for one day and then transplanted into kidney capsules preovulatory follicles were present at 15 days after transplantation before hCG treatment. Oocytes in preovulatory follicles showed intact germinal vesicles with surrounding cumulus cells (FIG. 7, upper right, GV stage oocyte, arrow). In contrast, ovaries from animals treated with hCG for 8 hours exhibited oocytes showing germinal vesicle breakdown (FIG. 7, lower left). These oocytes had no nuclear membrane but showed meiotic spindles (FIG. 7, lower right).

FIG. 8. Transplanted ovaries treated with the PTEN inhibitor in vitro (lower panel) showed clear increases in sizes as compared with corresponding control ovaries (upper panel) under a dissecting microscope.

FIG. 9. Diagrammatic representation of the follicle activation procedure. Paired ovaries from day 3 mice containing mainly primordial follicles were treated in vitro without or with PTEN inhibitor (bpV(pic)) and/or the PI3K activating peptide (740Y-P) for 2 days before transplantation into adult ovariectomized mice of the same strain. Hosts were then treated with FSH to promote follicle development.

FIG. 10. Activation of the PI3K-Akt-Foxo3 pathway in oocytes and increased follicle development after treatment with bpV(pic) and 740Y-P. (A) Nuclear exclusion of Foxo3 in oocytes of primordial follicles at 6 h after treatment with bpV(pic) and 740Y-P (insets: higher magnification). Bar=100 um. (B) Increases in the fraction of oocytes showing nuclear export of Foxo3. (C) AMH staining in granulosa cells of primary and secondary follicles at 2 days after in vitro culture. (D) BrdU incorporation into granulosa cells of activated follicles at 5 days after transplantation (top panels: hematoxylin and eosin staining; lower panels: BrdU staining). Bars=100 um.

FIG. 11. Activation of dormant primordial follicles after in vitro treatment with bpV(pic) and 740Y-P, followed by transplantation into the kidney capsule of ovariectomized hosts. (A) Ovaries at 14 days after transplantation (left panels, representative kidney pictures; right panels, isolated ovaries for control vs. bpV(pic), control vs. 740Y-P, and control vs. bpV(pic) plus 740Y-P groups. Bars=1 mm. (B) Ovarian weight increases as fold changes relative to paired non-treated controls. Numbers of paired ovaries used for analyses are shown in parentheses. (C) Ovarian histology showing follicle development to the large antral stage (arrow) at 14 days after treatment with bpV(pic) and 740Y-P followed by transplantation. Bars=200 um. (D) Distribution of follicles in ovaries without and with treatment with bpV(pic) and 740Y-P. Follicle dynamics of day 3 ovaries before transplantation is included for comparison. sec: secondary follicle. (E) Ovarian morphology at 14 days after transplantation of grafts treated with bpV(pic) and 740Y-P with or without inhibitors to Akt (SH5) or PI3K (Wortmannin). The symbol −/indicates groups without the inhibitors. Bar=1 mm.

FIG. 12. Retrieval of mature mouse oocytes for epigenetic analyses, in vitro fertilization and early embryonic development. (A) Histology of ovaries before and after hCG treatment at 18 days after transplantation following in vitro activation using bpV(pic) and 740Y-P. Left panels: an ovary before hCG treatment showing preovulatory follicles with an oocyte at the GV stage; Right panels: an ovary after hCG treatment showing preovulatory follicles with an oocyte exhibiting germinal vesicle breakdown. Bars=100 um. (B) Methylation status of the differentially methylated regions (DMRs) of two maternal imprinted genes, Igf2r and Lit1, and one paternal imprinted gene H19 in mature oocytes retrieved from transplanted ovaries after in vitro activation (T) and those from super-ovulated control ovaries (C). Each row represents a unique methylation profile within the pool of clones sequenced with the frequency of that methylation state indicated on the right side of each row. Each circle within the row represents a single CpG site (open circles, non-methylated cytosines; filled circles, methylated cytosines). N=15-20 sequenced clones. (C) Early embryonic development of retrieved oocytes after in vitro fertilization. Representative figures for embryos reaching 2-cell (24 h), 4-cell (48 h), morula (72 h), and blastocyst (96 h) stages. (D) Efficiency of early embryonic development. Percentage of mature oocytes retrieved from transplanted grafts capable of developing into 2-cell embryos and blastocysts as compared with mature oocytes obtained from ovaries from super-ovulated controls without transplantation.

FIG. 13. Activation of human primordial follicles from patients with benign ovarian tumor. (A) Left panels: Increased nuclear export of Foxo3 in primordial follicles after 1 h treatment with 100 uM bpV(pic). Right panel: Percentage of primordial oocytes showing Foxo3 nuclear export in control and bpV(pic)-treated groups. Bars=50 um. (B) Ovarian morphology at 6 months after xeno-transplantation into SCID mice (top panel: kidneys with ovarian grafts; lower panel: in situ kidney picture of one host). (C) Distribution of follicles at different stages in grafts with or without bpV(pic) treatment. Follicle distribution in cortical cubes before xeno-grafting is provided for comparison. sec: secondary follicles. (D) Representative sections showing the development of two large antral follicles in bpV(pic)-treated group with mature oocytes exhibiting germinal vesicle breakdown after hCG treatment (insets).

### DESCRIPTION OF THE SPECIFIC EMBODIMENTS

Compositions and methods are provided for modulating the survival and maturation of mammalian ovarian follicles. By selectively exposing follicles to an inhibitor of PTEN, follicle dormancy and consequent oocyte maturation can be manipulated. Compounds that inhibit PTEN are administered to an individual or provided to one or more ovarian follicles in vitro to release the follicles from dormancy. In some embodiments, the exposure to a PTEN inhibitor and/or PI3K activator is followed by exposure to one or more factors involved in oocyte maturation, including, without limitation, FSH or FSH analogs.

The methods of the invention find use in a wide variety of animal species, particularly including mammalian species. Animal models, particularly small mammals, e.g. murine, lagomorpha, etc. are of interest for experimental investigations. Other animal species may benefit from improvements in in vitro fertilization, e.g. horses, cattle, rare zoo animals such as panda bears, large cats, etc. Humans are of particular interest for enhancing oocyte maturation, including methods of in vitro fertilization. Individuals of interest for treatment with the methods of the invention include, without limitation, those suffering from premature ovarian failure, peri-menopause, etc.

In some embodiments of the invention, the exposure to a PTEN inhibitor and/or PI3K activator is performed in vitro, e.g. in an organ or tissue culture, where at least one primordial ovarian follicle is transiently exposed to an effective dose of a PTEN inhibitor and/or PI3K activator. The treated follicle may be utilized in vitro, for example for in vitro fertilization, generation of embryonic stem cells, etc., or may be transplanted to provide for in vivo uses.

In some embodiments, in vitro treatment with PTEN inhibitor and/or PI3K activators is followed by ovarian transplantation to activate primordial follicles for the generation of preovulatory oocytes, which may be followed by in vitro or in vivo fertilization. Individuals of interest include endangered species, economically important animals, women suffering from premature ovarian failure, follicles derived from human embryonic stem cells and primordial germ cells, and the like.

In other embodiments, the exposure to a PTEN inhibitor and/or PI3K activator is performed in vivo, where the PTEN inhibitor and/or PI3K activator may be locally or systemically administered to an individual. Following such exposure, the individual may be treated with FSH or FSH analogs, including recombinant FSH, naturally occurring FSH in an in vivo host animal, FSH analogs, e.g. FSH-CTP, pegylated FSH, and the like, at a concentration that is effective to release a mature oocyte.

Embodiments of the invention can include ovarian follicles of numerous species of mammals. The invention should be understood not to be limited to the species of mammals cited by the specific examples within this patent application. Embodiments of the invention, for example, may include fresh or frozen-thawed follicles of animals having commercial value for meat or dairy production such as swine, bovids, ovids, equids, buffalo, or the like (naturally the mammals used for meat or dairy production may vary from culture to culture). It may also include ovarian follicles from individuals having rare or uncommon attribute(s), such as morphological characteristics including weight, size, or conformation, or other desired characteristics such as speed, agility, intellect, or the like. It may include ovarian follicles from deceased donors, or from rare or exotic mammals, such as zoological specimens or endangered species. Embodiments of the invention may also include fresh or frozen-thawed ovarian follicles collected from primates, including but not limited to, chimpanzees, gorillas, or the like, and may also ovarian follicles from marine mammals, such as whales or porpoises.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Ovarian Follicle.

An ovarian follicle is the basic unit of female reproductive biology and is composed of roughly spherical aggregations of cells found in the ovary. A follicle contains a single oocyte. Follicles are periodically initiated to grow and develop, culminating in ovulation of usually a single competent oocyte. The cells of the ovarian follicle are the oocyte, granulosa cells and the cells of the internal and external theca layers. The oocyte in a follicle is in the stage of a primary oocyte. The nucleus of such an oocyte is called a germinal vesicle. Granulosa cells within the follicle surround the oocyte; their numbers increase in response to gonadotropins. They also produce peptides involved in ovarian hormone synthesis regulation. Follicle-stimulating hormone (FSH) acts on granulosa cells to express luteinizing hormone (LH) receptors on the cell surface. The granulosa cells, in turn, are enclosed in a thin layer of extracellular matrix—the follicular basement membrane or basal lamina (fibro-vascular coat in picture). Outside the basal lamina, the layers theca interna and theca externa are found.

Ovarian In Vitro Culture.

Methods are known in the art for culturing mammalian ovaries or fragments thereof, which fragments for the purposes of the present invention will include at least one follicle. Typically all or a portion of an ovary is placed in tissue culture medium, which medium may include factors useful in the growth or maintenance of the follicle cells, and may, as described herein, further comprise an effective dose of a PTEN inhibitor. See the Examples provided herein. Additional description may be found, inter alia, (each of which reference is herein specifically incorporated by reference) at Hoyer et al. (2007) Birth Defects Res B Dev Reprod Toxicol. 80(2):113-25. In vitro culture of canine ovaries is described by Luvoni et al. (2005) Theriogenology.; 63(1):41-59. Culture of bovine follicles is described by Hansel (2003) Anim Reprod Sci.; 79(3-4):191-201. Fortune (2002) Ernst Schering Res Found Workshop. (41):11-21 describes organ cultures using small pieces of ovarian cortex, or grafts of ovarian cortical pieces beneath the CAM of chick embryos.

A review of in vitro ovarian tissue and organ culture may be found in Devine et al. (2002) Front Biosci. 7:d1979-89; and in Smitz et al. (2002) Reproduction. 123(2):185-202. Whole ovaries from fetal or neonatal rodents have been incubated in organ culture systems. This has been utilized to understand the sequence of follicle formation and its hormonal requirements, activation of quiescent follicles, follicular growth and development, and acquisition of steroidogenic capabilities. Adaptations of this technique include incubation of ovaries in a chamber continuously perfused with medium or perfusion of medium through the intact vasculature. Late follicular development, ovulation, and steroidogenesis can also be examined in these systems. Another approach has been to culture individual follicles isolated by enzymatic or mechanical dissociation. Cryopreservation of human primordial and primary ovarian follicles is described by Hovatta (2000) Mol Cell Endocrinol. 169(1-2):95-7.

Ovarian Transplantation.

Starting in 1950s, ovarian transplantation to the kidney is a well-established procedure in animal studies. Later on, primordial follicles isolated from infant mouse ovaries by enzymatic digestion were transplanted into ovarian bursa of adult hosts sterilized by X-irradiation or ovariectomy. Ovaries forming from grafts were capable of spontaneous ovulation and the majority of animals carrying them were receptive to males. Mating often resulted in pregnancies and delivery of normal offspring. Furthermore, primordial follicles can be cryopreserved before transplantation. In women, successful ovarian transplantation between monozygotic twins discordant for premature ovarian failure has been reported. After unsuccessful egg-donation therapy, the sterile twin received a transplant of ovarian cortical tissue from her sister. After transplantation, the patient became pregnant and delivered a healthy baby. In addition to this case of ovarian transplantation, autologous transplantation of ovarian cortical strips to the forearm has been successfully performed in women undergoing sterilizing cancer therapy or surgery as demonstrated by the preservation of endocrine functions. Here, the ovarian transplantation approach may be used to activate dormant primordial follicles.

PTEN Inhibitor.

The polypeptide PTEN (phosphatase with TENsin homology) was identified as a tumor suppressor that is mutated in a large number of cancers at high frequency. The protein encoded this gene is a phosphatidylinositol-3,4,5-trisphosphate 3-phosphatase. It contains a tensin like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3,4,5-trisphosphate in cells and functions as a tumor suppressor by negatively regulating AKT/PKB signaling pathway. The genetic sequence of the human protein may be found in Genbank, accession number NM 000314, as described by Volinia et al. (2008) PLoS ONE 3 (10), E3380; Li et al. (1997) Cancer Res. 57(11), 2124-2129; Steck et al. (1997) Nat. Genet. 15(4), 356-362; and Li et al. (1997) Science 275 (5308), 1943-1947, each herein specifically incorporated by reference. PTEN inhibitors of interest may have an IC₅₀ of from about 0.1 nM to about 100 μM, and may be from about 1 nm to about 10 μM, of from about 10 nM to about 1 μM, of from about 1 nM to about 100 nM.

A number of known PTEN inhibitors are known in the art, including without limitation, bisperoxovanadium compounds (see, for example, Schmid et al. (2004) FEBS Lett. 566(1-3):35-8). Included are potassium bisperoxo(bipyridine)oxovanadate (V), which inhibits PTEN at an IC₅₀=18 nM; dipotassium bisperoxo(5-hydroxypyridine-2-carboxyl)oxovanadate (V), which inhibits PTEN at an IC₅₀=14 nM; potassium bisperoxo (1,10-phenanthroline)oxovanadate (V) which inhibits PTEN at an IC₅₀=38 nM; dipotassium bisperoxo(picolinato)oxovanadate (V) which inhibits PTEN at an IC₅₀=31 nM; N-(2-Hydroxy-3-methoxy-5-dimethylamino)benzyl, N′-(2-(4-nitrophenethyl)), N″-methylamine which inhibits the CDC25 phosphatase family; dephostatin which is a competitive PTP inhibitor; monoperoxo(picolinato)oxovanadate(V) which is a PTP inhibitor (IC₅₀=18 μM); and sodium orthovanadate, which is a broad-spectrum inhibitor of phosphatases.

Additional PTEN inhibitors are described by, inter alia, Myers et al. (1998) PNAS 95:13513-13518; by Garlich et al., WO/2005/097119; and by Rosivatz et al. (2007) ACS Chem. Biol., 1, 780-790.

Alternatively, inhibitors of PTEN may be identified by compound screening for agents, e.g. polynucleotides, antibodies, small molecules, etc., that inhibit the enzymatic activity of PTEN, which is known to have phosphatase activity. Compound screening may be performed using an in vitro model, a genetically altered cell or animal or purified PTEN1 protein. One can identify ligands or substrates that bind to or inhibit the phosphatase activity. A wide variety of assays may be used for this purpose, including labeled in vitro protein-protein binding assays, electrophoretic mobility shift assays, immunoassays for protein binding, and the like. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Antibodies specific for PTEN or epitopic fragments thereof may be used in the methods of the invention. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a PTEN polypeptide.

Antibodies are prepared in accordance with conventional ways, where the expressed polypeptide or protein is used as an immunogen, by itself or conjugated to known immunogenic carriers, e.g. KLH, pre S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

PI3K activator. Phosphoinositide 3-kinases (PI 3-kinases or PI3Ks) are a family of enzymes involved in cellular functions such as cell growth, proliferation, differentiation, motility, survival and intracellular trafficking, which are capable of phosphorylating the 3 position hydroxyl group of the inositol ring of phosphatidylinositol (PtdIns).

Class I PI3Ks are responsible for the production of Phosphatidylinositol 3-phosphate (PI(3)P), Phosphatidylinositol (3,4)-bisphosphate (PI(3,4)P₂) and Phosphatidylinositol (3,4,5)-trisphosphate (PI(3,4,5)P₃. The PI3K is activated by G-protein coupled receptors and tyrosine kinase receptors.

Class I PI3K are heterodimeric molecules composed of a regulatory and a catalytic subunit; which are further divided between IA and IB subsets on sequence similarity. Class I PI 3-kinases are composed of a catalytic subunit known as p110 and a regulatory subunit either related to p85 or p101. The p85 subunits contain SH2 and SH3 domains.

Activators of PI3K increase the activity of the enzyme. Activators of interest include, without limitation the cell-permeable phospho-peptide (740Y-P), which is capable of binding to the SH2 domain of the p85 regulatory subunit of PI3K to stimulate enzyme activity (commercially available peptide, RQIKIWFQNRRMKWKKSDGGYMDMS, Modifications: Tyr-25=pTyr). Other activators include fMLP (see Inoue T, Meyer T (2008) Synthetic Activation of Endogenous PI3K and Rac Identifies an AND-Gate Switch for Cell Polarization and Migration. PLoS ONE 3(8): e3068. Also see Bastian et al., Mol Cancer Res 2006; 4(6). June 2006; Park et al. Toxicology Toxicology Volume 265, Issue 3, 30 Nov. 2009, Pages 80-86, herein incorporated by reference)

FSH.

Follicle-stimulating hormone (FSH) is a hormone synthesized and secreted by gonadotropes in the anterior pituitary gland. FSH regulates the development, growth, pubertal maturation, and reproductive processes of the human body. FSH and Luteinizing hormone (LH) act synergistically in reproduction. In females, in the ovary FSH stimulates the growth of immature Graafian follicles to maturation. Graafian follicles are the mature preovulatory follicle. Primary follicles mature to Graafian follicles. As the follicle grows, it releases inhibin, which shuts off the FSH production.

FSH is a dimeric glycoprotein. The alpha subunits of LH, FSH, TSH, and hCG are identical, and contain 92 amino acids. FSH has a beta subunit of 118 amino acids (FSHB), which confers its specific biologic action and is responsible for interaction with the FSH-receptor. The half-life of native FSH is 3-4 hours. Its molecular wt is 30000.

Various formulations of FSH are available for clinical use. It is used commonly in infertility therapy to stimulate follicular development, notably in IVF therapy, as well as with interuterine insemination ° Up. FSH is available mixed with LH in the form of Pergonal or Menopur, and other more purified forms of urinary gonadotropins, as well as in a pure forms as recombinant FSH (Gonal F, Follistim), and as Follistim AQ, Gonal-F, Gonal-f RFF, Gonal-f RFF Pen.

Analogs of FSH are also clinically useful, which analogs include all biologically active mutant forms, e.g. where one, two, three or more amino acids are altered from the native form, PEGylated FSH, single chain bi-functional mutants, FSH-CTP, and the like. In an effort to enhance ovarian response several long-acting FSH therapies have been developed including an FSH-CTP (Corifollitropin alfa), where the FSH subunits are linked by the C-terminal peptide (CTP) moiety from human chorionic gonadotropin (hCG); and single-chain bi-functional VEGF-FSH-CTP (VFC) analog. FSH-CTP has a longer half-life in vivo, and may be administered, for example, with an interval of from one to four weeks between doses. See, for example, Lapolt et al. (1992) Endocrinology 131:2514-2520; and Devroey et al. (2004) The Journal of Clinical Endocrinology & Metabolism Vol. 89, No. 5 2062-2070, each herein specifically incorporated by reference.

Candidates for Therapy.

Any female human subject who possesses viable ovarian follicles is a candidate for therapy with the methods of the invention. Typically, the subject will suffer from some form of infertility, including premature ovarian failure. For instance, the subject may experience normal oocyte production but have an impediment to fertilization, as in, e.g. PCOS or PCOS-like ovaries. The methods of the invention may be especially useful in women who are not suitable candidates for traditional in vitro fertilization techniques involving an ovarian stimulation protocol.

As described above, the methods of the invention are also useful in the treatment of infertility with various non-human animals, usually mammals, e.g. equines, canines, bovines, etc.

Premature ovarian failure (POF) occurs in 1% of women. The known causes for POF include genetic aberrations involving the X chromosome or autosomes as well as autoimmune ovarian damages. Presently, the only proven means for infertility treatment in POF patients involve assisted conception with donated oocytes. Although embryo cryopreservation, ovarian cryopreservation, and oocyte cryopreservation hold promise in cases where ovarian failure is foreseeable as in women undergoing cancer treatments, there are few other options. Due to heterogeneity of POF etiology, varying amounts of residual primordial follicle are still present in patients' ovaries for activation by PTEN inhibitors.

The degrees of ovarian follicle exhaustion vary among POF patients. The methods of the present invention allow the activation of the remaining primordial follicles in POF patients using PTEN inhibitors, followed by ovarian transplantation and FSH or FSH analog treatment to promote the development of early follicles to the preovulatory stage. This may be followed by the retrieval of mature oocytes for IVF and subsequent pregnancy following embryo transfer. Due to the delay of child-bearing age in the modern society, many perk menopausal women also are experiencing infertility as the result of diminishing ovarian reserve. Although gonadotropin treatments are widely used to promote the development of early antral follicles to the preovulatory stage, many peri-menopausal patients do not respond to the gonadotropin therapy. Because these women still have varying numbers of primordial follicles, they also benefit from the methods of the invention.

### Methods of Enhancing Ooocyte Maturation

Methods are provided for activating dormant ovarian primordial follicles in a mammal to promote development to preovulatory follicles. In the methods of the invention, ovarian follicles are transiently exposed in vitro or in vivo to an inhibitor of PTEN and/or an activator of PI3K at a concentration that is effective to induce the follicles to initiate growth, i.e. to break dormancy. Following exposure to a PTEN inhibitor and/or PI3K activator, the follicle may be exposed to follicle stimulating hormone (FSH or FSH analog) to provide for gonadotropin stimulation.

In some embodiments of the invention, the exposure to a PTEN inhibitor and/or PI3K activator is performed in vitro. In such embodiments, an ovary or a fraction thereof comprising at least one follicle is obtained from a suitable mammalian donor and maintained in culture in vitro. The tissue in culture is contacted with an effective dose of a PTEN inhibitor and/or PI3K activator, which inhibitors have been described above. The dose of inhibitor is sufficient to release the follicles from dormancy, and as such, will vary according to the specific inhibitor that is used, the length of time it is provided in the culture, the condition of the follicles, etc. Methods known in the art for empirical determination of concentration may be used. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

As an example, follicle cultures may be contacted with dipotassium bisperoxo(picolinato) oxovanadate (V) at a final concentration of at least about 1 μM, at least about 25 μM, at least about 100 μM, at least about 250 μM and not more than about 1 mM, for a transient period of time of at least about 1 hour to about 24 hours, and may be from about 6 to about 12 hours. The concentrations may be adjusted to reflect the potency of other inhibitors.

Following release of follicles from dormancy, the oocytes present in the follicles may be utilized for in vitro purposes. In some embodiments the oocytes are utilized directly, and in others the follicles are contacted with one or more factors to modulate the oocyte maturation, e.g. the cultures may be contacted with a concentration of FSH or FSH analog sufficient to induce oocyte maturation in vitro, where the FSH or FSH analog may be recombinant, modified, native, etc. Following in vitro maturation the oocytes may be fertilized in vitro for implantation; may be fertilized in vitro for generation of stem cell lines; may be utilized without fertilization for various research purposes, and the like.

The follicles may be additionally cultured in the presence of one or more of c-kit ligand (Hutt et al., 2006; Parrott and Skinner, 1999), neurotrophins (Ojeda et al., 2000), vascular endothelial growth factor (Roberts et al., 2007), bone morphogenetic protein (BMP)-4 (Tanwar et al., 2008), BMP7 (Lee et al., 2001), leukemia inhibitory factor (Nilsson et al., 2002), basic FGF (Nilsson et al., 2001), keratinocyte growth factor (Kezele et al., 2005), and the like, where the factor(s) may be added with a PTEN inhibitor and/or PI3K activator, or following exposure to the PTEN inhibitor and/or PI3K activator.

In other embodiments the follicles may be transplanted to an animal recipient for maturation. As described above, methods are known in the art for transplantation of ovaries or fragments thereof at an ovarian site, at a kidney site, at a sub-cutaneous site, etc. are known in the art and may find use. Where the ovarian tissue is transplanted to an ovary, fertilization may proceed without additional in vitro manipulation. Where the ovarian tissue is transplanted to a non-ovarian site, e.g. a sub-cutaneous site, the oocytes may be subsequently removed for in vitro fertilization. The recipient may provide endogenous FSH for maturation of the oocytes, or may be provided with exogenous FSH or FSH analog for that purpose, including recombinant, long-acting FSH-CTP, and the like.

In other embodiments, the exposure to a PTEN inhibitor and/or PI3K activator is performed in vivo, where the PTEN inhibitor and/or PI3K activator may be locally or systemically administered to an individual. The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized. The individual is typically contacted with a PTEN inhibitor and/or PI3K activator at an effective concentration for at least about 6 hours, usually at least about 12 hours, and may be for at least about 1 day and not more than about one week, usually not more than about 3 days.

The PTEN inhibitor and/or PI3K activator compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The PTEN inhibitor and/or PI3K activator can be administered in a variety of different ways. Examples include administering a composition via oral, topical, intraperitoneal, intravenous, intramuscular, subcutaneous, subdermal, transdermal, intra-ovarian methods. In pharmaceutical dosage forms, the compounds may be administered in the form of their pharmaceutically acceptable salts, or they may also be used alone or in appropriate association, as well as in combination with other pharmaceutically active compounds.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

Following such exposure, the individual may be treated with recombinant FSH or FSH analogs, including, without limitation, naturally occurring FSH in an in vivo host animal, FSH analogs such as FSH-CTP, single chain analogs, pegylated FSH, and the like, at a concentration that is effective to release a mature oocyte. Alternatively, the oocytes may be removed from the ovary and utilized for in vitro manipulation as described above.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

Deletion of FOX3A1 or PTEN in the PI3K-PKB/Akt Pathway LED to Premature Ovarian Failure in Mutant Mice:

Because the diverse local hormones/factors involved in initial follicle recruitment are likely to converge on the same intracellular signaling pathways, it is easier to manipulate the functions of intracellular genes for the activation of dormant primordial follicles. Recent studies provide insights into the intracellular mechanisms important for primordial follicle activation from the dormant state. The phosphatidylinositol 3-kinase (PI3K) signalling pathway begins with PI3K activation by receptor tyrosine kinases. PI3K phosphorylates and converts the lipid second messenger phosphatidylinositol (4,5) bisphosphate (PIP2) into phosphatidylinositol (3,4,5) triphosphate (PIP3), which recruits and activates phosphatidylinositol-dependent kinase 1 (PDK1). PDK1, in turn, phosphorylates and activates PKB (also known as AKT) which inhibits the activities of the forkhead (Foxo) transcription factors, resulting in cell proliferation and survival.

In addition to the activation of PKB/Akt by PI3K, this important pathway is also regulated by an inhibitor. The tumor-suppressor Phosphatase with tensin homology (PTEN) protein negatively regulates PI3K signalling by dephosphorylating PIP3, converting it back to PIP2. The PTEN gene was cloned as a candidate tumor suppressor gene from the chromosome 10q23 region, a locus frequently targeted for genetic loss in tumors (Li et al. (1997) Science 275:1943-1947). Somatic inactivation of both PTEN alleles and loss of heterozygosity have been demonstrated in a number of tumors including glioblastoma, melanoma, and prostate, breast, and endometrial carcinomas.

The PI3K-PKB/Akt signaling pathway is important for the proper function of all cells in the body. In ovarian follicles, the PKB/Akt pathway is obligatory for the FSH-induced granulosa cell differentiation (Zeleznick et al. (2003) Endocrinology 144:3985-3994) whereas PKB/Akt is important for oocyte maturation (Kalous et al. (2006) Biol. Cell 98:111-123). Immunoblotting analyses further demonstrated the expression of Akt and upstream PDK1 in mature oocytes. In the sheep ovary, PTEN was found in granulosa cells of large differentiated follicles. In human ovaries, immunoreactive PKB/Akt was found in the oocytes, granulosa cells, and theca cells of primordial and growing follicles (Goto et al. (2007) J Assist Reprod Genet. 24:541-6). As the follicles grew, staining for PTEN became intense in the granulosa cells. In the oocytes of some follicles, PTEN staining was also evident. These data suggested that PTEN and PKB/Akt are present in the granulosa cells during folliculogenesis. An increase in PTEN may lead to changes in proliferation and/or differentiation of granulosa cells during follicular growth via regulation of Akt phosphorylation. Furthermore, key genes in the PTEN-PDK1-PKB/Akt pathway are expressed in the oocyte.

The activity of a group of Foxo transcriptional factors are inhibited by PKB/Akt and the Foxo genes are essential for maintaining cell-cycle arrest. Activation of the Akt/PKB pathway led to the suppression of Foxo transcriptional activities, leading to cell growth. Although the expression of these Foxo genes in the oocyte is not clear, an interesting study indicated that disruption of one of the Foxo transcriptional factors, Foxo3A, led to the activation of all dormant primordial follicles (Castrillon et al. (2003) 301:215-8; and John et al (2008) Dev Biol. 321:197-204; and John et al (2007) Reproduction 133:855-63.). The Foxo3A null female mice exhibited global follicular activation, followed by early depletion of functional ovarian follicles, and secondary infertility. Foxo3A was proposed to function at the earliest stage of follicular growth as a suppressor of follicular activation.

Following findings of ovarian infertility in Foxo3A null mice, Reddy et al hypothesized that activation of the PKB/Akt pathway in the oocyte could also suppress Foxo3A activity to allow the activation of dormant primordial follicles (Reddy et al. (2008) Science 319:611-613). Increases in oocyte PKB/Akt activity were achieved by oocyte-specific deletion of the PTEN enzyme so that PIP3 cannot be efficiently degraded, leading to the activation of the PI3K-PKB/Akt pathway and Foxo3A suppression. These authors generated transgenic crosses of PTEN-loxP and GDF9 promoter-driven Cre mice to specifically delete oocyte PTEN expression of early follicles. Similar to the Foxo3A null mice, the entire primordial follicle pool in these transgenic mice became activated after PTEN deletion in the oocyte, followed by the exhaustion of the primordial follicle reserve and premature ovarian failure.

Inhibition of PTEN activity using vanadium derivatives.

In addition to the role of PTEN in cell proliferation and tumorigenesis, the loss or impairment of PTEN results in an antidiabetic impact. Systemic administration of PTEN antisense oligonucleotide once a week in mice suppressed PTEN expression in liver and fat, and normalized blood glucose concentrations in db/db and ob/ob mice. These findings led to the suggestion that PTEN could be an important target for drugs against type II diabetes. Vanadate is a competitive reversible inhibitor for protein tyrosine phosphatases (PTPases). In particular, peroxovanadium (pV) compounds activate the insulin receptor kinase in hepatocytes and inhibit the dephosphorylation of insulin receptors in hepatic endosomes. These vanadate derivatives such the bisperoxovanadium (bpV) have been employed as PTPase inhibitors and insulin mimetics. Given that PTPases and PTEN share considerable homology in their active site, several groups demonstrated that the characterized PTPase inhibitors, bpV compounds, can also inhibit the PI3K-phosphatase PTEN. Testing several different compounds in vitro revealed that bpVs with polar N,O ligands (e.g. bpV(pic)) had a strong preference towards PTEN (IC₅₀: 20-40 nM) and inhibited PTEN at 10- to 100-fold lower concentrations than PTPases. These small molecular weight compounds, including bpV(pic), increased cellular PIP3 levels, phosphorylation of Akt, and glucose uptake in adipocytes at nanomolar concentrations. Of interest, intravenous administration of bpV(pic) stimulated [14C]glucose incorporation into rat diaphragm glycogen and augmented rat diaphragm insulin receptor kinase activity.

Exposure to PTEN Inhibitors Stimulates the Initial Recruitment of Dormant Primordial Follicles, In Vitro Exposure of Neonatal Ovaries to PTEN Inhibitors Followed by Transplantation into the Kidney.

We incubated ovaries with 250 μM bpV(pic) (Calbiochem, San Diego, Calif.) for 6 h to activate primordial follicles. The dosage of the PTEN inhibitor is decreased by using 100 and 30 μM of the compound to determine if it is still capable of activating the primordial follicles. The duration of the inhibitor treatment is decreased and increased by incubating the ovaries for 3 and 12 h to determine the extent of dormant follicle activation. The percentage of primordial, primary, preantral, and larger follicles after ovarian transplantation is monitored as described below.

In Vivo Transplantation to Promote Preovulatory Follicle Development.

The established kidney transplantation approach was used to allow follicle development in vivo. Daily injection of FSH was used to promote follicle development after transplantation into adult hosts ovariectomized immediately before ovarian transplantation. We chose a dose of 2 IU/25 g body weight of FSH based on earlier studies in neonatal rats. We used ovariectomized hosts because the endogenous gonadotropins (both FSH and LH) are elevated in POF patients and peri-menopausal women. The present injection of hosts with FSH maintains a high FSH/LH ratio because our studies indicated that increases in LH/FSH ratio could lead to premature luteinization of follicular cells.

Ovaries fixed for at least 24 h are dehydrated, embedded in paraffin, and serially sectioned at 5 μm intervals. The sections are mounted on glass slides and stained with Mayers hematoxylin and eosin. Follicles are counted using the dissector and fractionator principles (see Mayhew et al. (1991) Exp Physiol 76:639-65). Every fifth section of each ovary is used for analysis. Follicle stages are determined based on the classification used by Flaws et al. (1997) Biol. Reprod. 57:1233-1237. Follicles are assessed blindly and confirmed by two investigators. Data is expressed as percentage of follicle per ovary.

Follicles are identified as healthy if they contained an intact oocyte, organized granulosa and thecal cell layers, and no pyknotic bodies. Follicles are considered atretic if they contain a degenerating oocyte, disorganized granulosa cells with pyknotic nuclei or apoptotic bodies. Follicles will be scored as “primordial” if they contain an intact oocyte with a visible nucleolus surrounded by a single layer of fusiform-shaped granulosa cells. Follicles are scored as “primary” if they consist of an intact, enlarged oocyte with a visible nucleolus and a single layer of cuboidal granulosa cells. Follicles are scored as preantral if they contain an oocyte with a visible nucleolus and more than one layer of granulosa or thecal cells. To obtain an estimate of the total number of follicles per ovary, the number of primordial, primary, and preantral follicles are multiplied by 5 to account for the fact that every fifth section was used for analyses. Statistical analysis of follicle number is performed using Sigmastat statistical software v.2 (Jandel Corporation, San Rafael, Calif., USA).

### Example 2

As described below, we promoted the initiation of primordial follicles by using PTEN inhibitors followed by in vivo treatment with FSH to accelerate the development of primary and secondary follicles to the early antral and preovulatory stages.

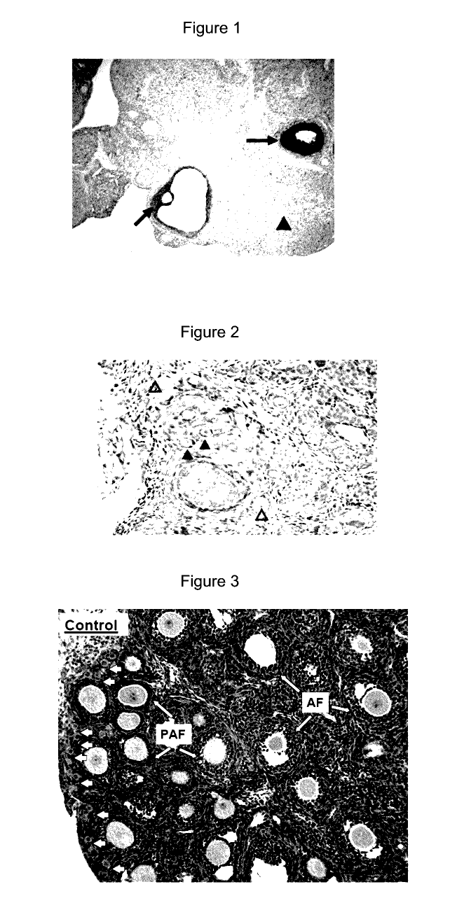

Expression of PTEN in oocyte and granulosa cells. Although oocyte-specific deletion of the PTEN gene led to the activation of all dormant primordial follicles, the expression of PTEN in the oocyte of primordial rodent follicles have not been studied. We performed immunohistochemical staining using ovaries from 9-weeks-old female rats. As shown in FIG. 1, strong PTEN staining was observed in cumulus and granulosa cells of preovulatory and antral follicles (arrows) whereas the luteal cells were weakly stained (arrowhead). Minimal PTEN staining was found in oocytes of these follicles. These findings in rodents are consistent with reports using ovine and human ovaries. Under a higher magnification, PTEN staining was detectable in oocytes of some primordial follicles (FIG. 2, solid arrowheads). However, PTEN staining was weaker for other primordial or primary follicles (FIG. 2, unfilled arrowheads). Assuming follicles expressing high levels of PTEN in their oocytes are arrested by this phosphatase, they can be recruited into the growing pool following treatment with PTEN inhibitors.

Transient Exposure of Ovaries to PTEN Inhibitor In Vitro LED to In Vivo Growth of Primordial Follicles.

Transient incubation of neonatal ovaries in vitro with bpV(pic) may avoid the adverse side effects of the PTEN inhibitor in vivo to allow a tissue-specific action of the inhibitor. After treatment using the PTEN inhibitor, ovaries are transplanted into adult hosts. To facilitate follicle growth, adult recipients are ovariectomized to increase endogenous gonadotropin levels. Furthermore, the hosts are treated with FSH to promote follicle development. Ovaries containing mainly primordial and primary follicles were obtained from Balb/c mice at day 3 of age (step 1). Pairs of ovaries were incubated with the PTEN inhibitor bpV(pic) (250 uM) or culture media (control group) for 6 h to regulate the initiation of primordial follicle growth (step 2). The paired ovaries (PTEN inhibitor-treated and controls) were then transplanted under two separate sides of the kidney capsule of the same ovariectomized adult (8-weeks-old) host (step 3). One day after transplantation, the hosts received daily i.p. injections of human FSH (2 IU/25 g body weight) for 12 days (step 4). At the end of the experiment, ovaries were collected from the kidney capsule and fixed for histological evaluation.

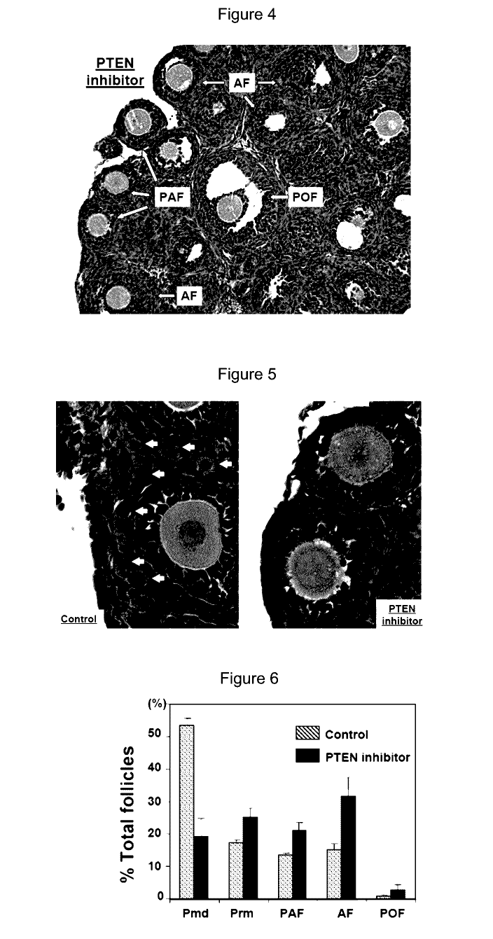

As shown in FIG. 3, control ovaries showed some follicles progressed to preantral follicle (PAF) and antral follicle (AF) stages but with many primordial follicles (white arrowheads) clustered near the cortex region. Follicles entering the growing pool were located in the medulla region. In PTEN inhibitor-treated ovaries (FIG. 4), most follicles entered the growing pool as reflected by the presence of many preantral follicles (PAF), antral follicles (AF) and a few preovulatory follicles (POF). These growing follicles were distributed in both medulla and cortical regions. In addition, few primordial follicles were found in the cortical or other region of the PTEN inhibitor-treated ovaries. Under a higher magnification, clusters of primordial follicles were evident in the control group (FIG. 5, left panel, white arrowheads) but few primordial follicles are present in ovaries treated with the PTEN inhibitor (FIG. 5, right panel). We performed preliminary analysis to estimate follicles at different developmental stages. Three pairs of ovaries were counted (˜500 follicles counted per ovary) using random sections and follicles at different developmental stages expressed as percentage of total. As shown in FIG. 6, treatment with the PTEN inhibitor decreased by 60% the percentage of primordial (Pmd) follicles as compared to controls. This was accompanied by increases in the percentage of primary (Prm), preantral (PAF), antral (AF), and preovulatory (POF) follicles in the PTEN inhibitor group. These data suggested that treatment with the PTEN inhibitor activated dormant primordial follicles to enter the growing pool.

We generated new data showing the induction of oocyte maturation in preovulatory follicles of transplanted ovaries after transient in vitro exposure to PTEN inhibitors. Neonatal ovaries were obtained from mice at 3 days of age and treated with the PTEN inhibitor bpV(pic) for 6 h. These ovaries were then transplanted under the kidney capsule of ovariectomized adult hosts. One day after transplantation, hosts received daily i.p. injections of human FSH (2 IU/25 g body weight). At 15 days after transplantation, ovariectomized hosts were injected with an ovulatory dose (7 IU) of hCG. Ovaries were then collected at 8 hours after hCG treatment for histological analyses. In some animals, ovaries were collected before hCG treatment. Similar results were obtained from 3 ovaries per group.

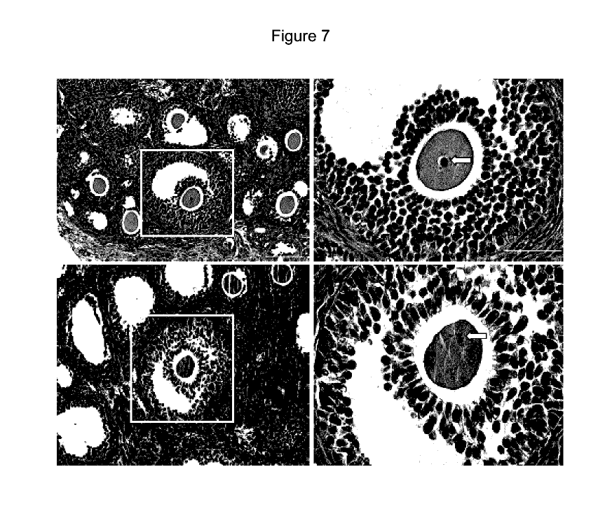

As shown in FIG. 7 (upper left), preovulatory follicles were present at 15 days after transplantation before hCG treatment. Oocytes in preovulatory follicles showed intact germinal vesicles with surrounding cumulus cells (FIG. 7, upper right, GV stage oocyte, arrow). In contrast, ovaries from animals treated with hCG for 8 hours exhibited oocytes showing germinal vesicle breakdown (FIG. 7, lower left). These oocytes had no nuclear membrane but showed meiotic spindles (FIG. 7, lower right). Furthermore, cumulus cell expansion took place in these preovulatory follicles as reflected by the presence of loosely associated cumulus cells. These findings demonstrated that the present transient exposure to the PTEN inhibitor before ovarian transplantation did not cause detrimental effects on follicle development and nuclear maturation of the preovulatory oocyte. The present kidney transplantation and FSH treatment protocol allows the maturation of preovulatory follicles containing oocytes capable of responding to the LH/hCG surge.

These methods are optimized for the retrieval of preovulatory oocytes from hCG-treated ovaries using needle puncture with or without collagenase treatment. Mature oocytes obtained from ovaries treated with the PTEN inhibitor are used for subsequent in vitro fertilization and embryo transfer experiments.

### Example 3

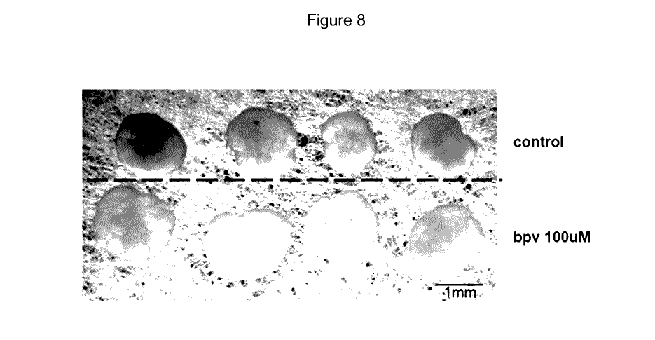

To further test the possibility of using lower doses of the PTEN inhibitor to activate dormant follicles, ovaries from 3-days-old mice were treated with 100 μM of bpV(pic) for 24 h before transplantation to kidney capsules of adult ovariectomized, FSH-treated recipients. Twelve days later, paired ovaries (control and PTEN inhibitor-treated) transplanted to each side of the kidney capsule of the same adult recipients were dissected out and layered on a wet towel. As shown in FIG. 8, ovaries treated with the PTEN inhibitor (lower panel) showed clear increases in sizes as compared with corresponding control ovaries (upper panel) under a dissecting microscope.

### Example 4

Although multiple follicles are present in mammalian ovaries, most of them remain dormant. During reproductive life, some follicles are activated for development. Transgenic mice with oocyte-specific deletion of genes in the PTEN-PI3K-Akt-Foxo3 pathway exhibited premature activation of all dormant follicles. Using an inhibitor of the PTEN phosphatase and a PI3K activating peptide, we found that short-term treatment of neonatal mouse ovaries increased nuclear exclusion of Foxo3 in primordial oocytes. After transplantation under kidney capsules of ovariectomized hosts, treated follicles developed to the preovulatory stage with mature eggs displaying normal methylation of imprinted genes. Following fertilization, healthy pups were delivered. Human ovarian cortical fragments from cancer patients were also treated with the PTEN inhibitor. After xeno-transplantation to immune-deficient mice for 6 months, primordial follicles developed to the preovulatory stage with oocytes capable of undergoing nuclear maturation. Major differences between male and female mammals are unlimited number of sperm and paucity of mature oocytes. We demonstrated that short-term stimulation of the PI3K-Akt pathway, followed by transplantation, is sufficient to activate dormant follicles to generate a large supply of mature female germ cells for future treatment of infertile women with a diminishing ovarian reserve and for cancer patients with cryo-preserved ovaries.

Studies using mutant mice indicated that oocyte-specific deletion of the PTEN gene promotes the growth of all primordial follicles in neonatal and adult animals. The PTEN gene encodes a phosphatase enzyme that negatively regulates the PI3K-Akt signaling pathway. Deletion of PTEN in the oocyte increases Akt phosphorylation and nuclear export of downstream Foxo3 proteins. Indeed, Foxo3 gene deletion also activated all dormant primordial follicles in mice. Here, we treated neonatal mouse ovaries in vitro with a PTEN inhibitor and a PI3K activating peptide to activate dormant primordial follicles, followed by transplanting them into the kidney capsule of FSH-treated adult ovariectomized recipients to promote follicle growth. We generated mature eggs capable of developing into viable and fertile offspring. Using human cortical strips containing primordial follicles, we also activated dormant human follicles to develop into large antral follicles containing mature oocytes after xeno-transplantation in immune-deficient mice.

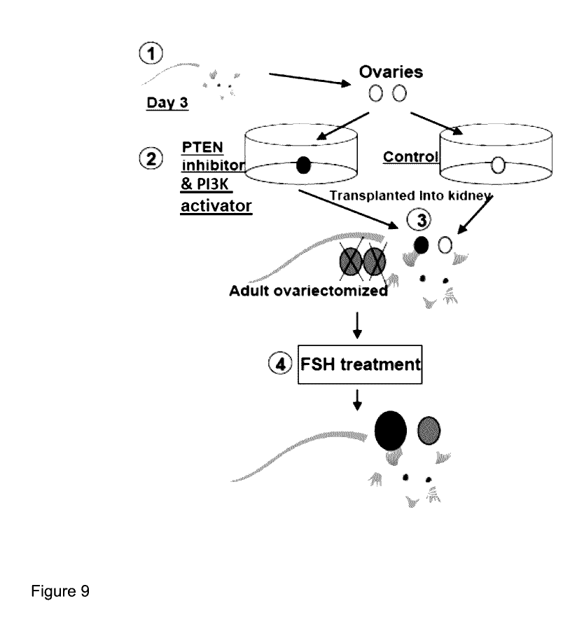

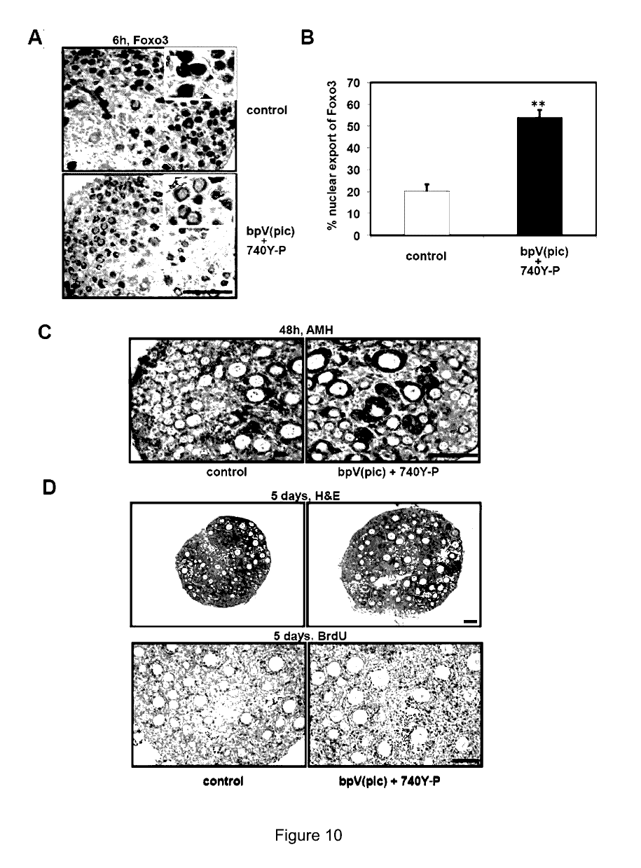

We hypothesized that transient incubation of neonatal mouse ovaries in vitro with bpV(pic), a PTEN inhibitor, allows the activation of dormant follicles. Ovaries containing mainly primordial follicles were obtained from neonatal mice at day 3 of age (FIG. 9, step 1). Pairs of ovaries were incubated with bpV(pic) (100 μM) or culture media (control group) for 24 h (step 2). In addition to bpV(pic), some ovaries were treated for 48 h with a cell-permeable phospho-peptide (740Y-P) capable of binding to the SH2 domain of the p85 regulatory subunit of PI3K to stimulate enzyme activity. Activated PI3K converts PIP2 (phosphatidylinositol (4, 5)-bisphosphate (PIP2)) to PIP3 (phosphatidylinositol (3,4,5)-trisphosphate (PIP3)) whereas the PTEN inhibitor prevents the conversion of PIP3 back to PIP2. Accumulated PIP3, in turn, could stimulate the phosphorylation of Akt and increase the nuclear exclusion of the transcriptional factor Foxo3. As shown in FIG. 10A, treatment of neonatal ovaries with bpV(pic) and the PI3K activating peptide increased the nuclear export of Foxo3 in oocytes of primordial follicles at 6 h after incubation. Quantitative analyses indicated that 54% of oocytes in primordial follicles exhibited Foxo3 export (FIG. 10B). At 48 h after treatment, granulosa cells of growing follicles in the treatment group showed increased staining of anti-Mullerian hormone (AMH), a marker for secondary/preantral follicles (FIG. 10C).

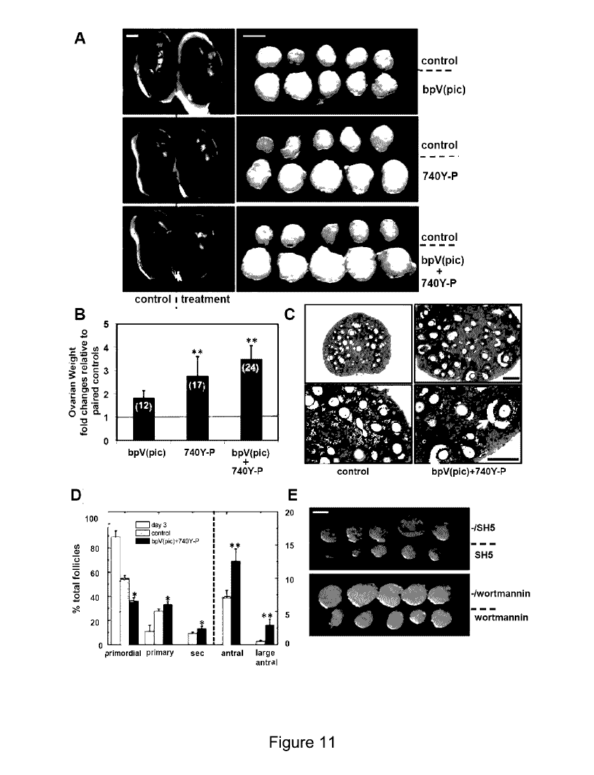

Paired ovaries (treated and untreated) from the same donor were then transplanted under separate sides of the kidney capsule in the same ovariectomized adult (8-weeks-old) recipient (FIG. 9, step 3). One day after transplantation, hosts received daily i.p. injection of FSH (2 IU/day) to promote follicle development. At five days after transplantation, bromodeoxyuridine (BrdU) labeling analyses showed increased proliferation of granulosa cells in growing follicles of treated ovaries as compared with controls (FIG. 10D). At 14 days after transplantation, increases in ovarian sizes of treated groups were evident as compared with paired controls (FIG. 11A); co-treatment of both bpV(pic) plus 740Y-P showed most pronounced increases based on ovarian weight determination (FIG. 11B), reaching 3.4-fold of the control level. Histological analyses further demonstrated the presence of large antral follicles (>250 um in diameter) in ovaries treated with bpV(pic) plus 740Y-P as compared with controls (FIG. 11C). Follicle counting of serial ovarian sections indicated 1.8- and 6-fold increases between control and treatment groups in the number of antral and large antral follicles, respectively (FIG. 11D). Also, in vitro co-treatment with bpV(pic) plus 740Y-P with or without inhibitors to Akt (SH-5) or PI3K (Wortmannin) demonstrated the mediatory roles of these enzymes in follicle activation (FIG. 11E).

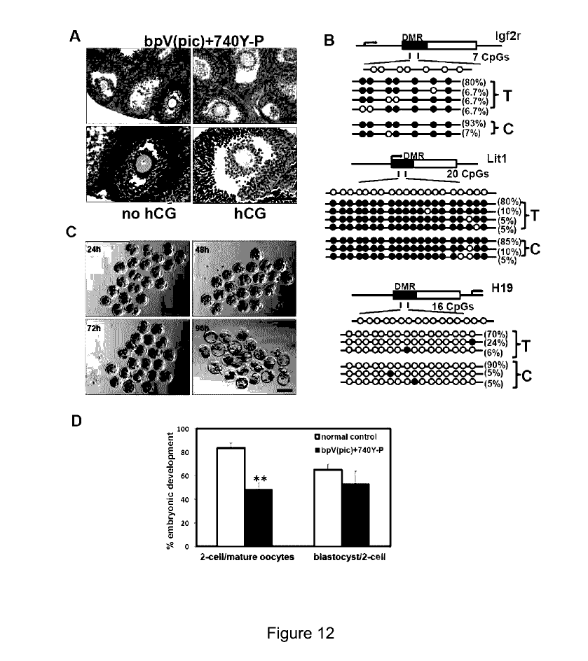

To test the maturity of oocytes from activated follicles, neonatal ovaries treated with bpV(pic) plus 740Y-P were transplanted for 18 days with FSH treatment. At 8 h before sacrifice, hosts received saline or an ovulatory dose of hCG to evaluate nuclear maturation of oocytes. As shown in FIG. 12A (upper left), large antral follicles were present in saline-treated (no hCG) animals. Oocytes in these follicles showed intact germinal vesicles and compact cumulus cells (lower left). In contrast, ovaries from hCG-treated group contained oocytes showing germinal vesicle breakdown (upper right) in preovulatory follicles. These oocytes had no nuclear membrane but showed meiotic spindles (lower right) and were surrounded by loosely associated cumulus cells, representing cumulus expansion. Follicle counting indicated that 31±4% of oocytes in antral follicles underwent nuclear maturation after hCG treatment. We further isolated mature oocytes from activated follicles at 18 days after transplantation to evaluate their developmental potential. Maternal-specific methylation of imprinted genes is established during ovarian follicle development. In mature oocytes (FIG. 12B), methylation patterns in the differential methylated regions (DMR) of two maternal imprinted genes, Igfr2 and Lit1, are comparable between those obtained after transplantation (T) or from super-ovulated control mice (C). For both groups, a paternal imprinted gene H19 showed minimal methylation. After in vitro fertilization using donor sperm, mature oocytes from transplanted ovaries developed into two-cell embryos (24 h), 4-cell embryos (48 h), morula (72 h), and blastocysts (96 h) during culture (FIG. 12C). Although the fraction of mature oocytes developing into two-cell embryos was lower for oocytes from transplanted ovaries than those from super-ovulated ovaries without transplantation (control), similar percentage of 2-cell embryos progressed to blastocysts for both groups (FIG. 12D). A total of 86 two-cell embryos were transferred into 6 pseudo-pregnant females, leading to the delivery of 12 healthy pups capable of developing into adults. These progeny exhibited normal mating and fertility.

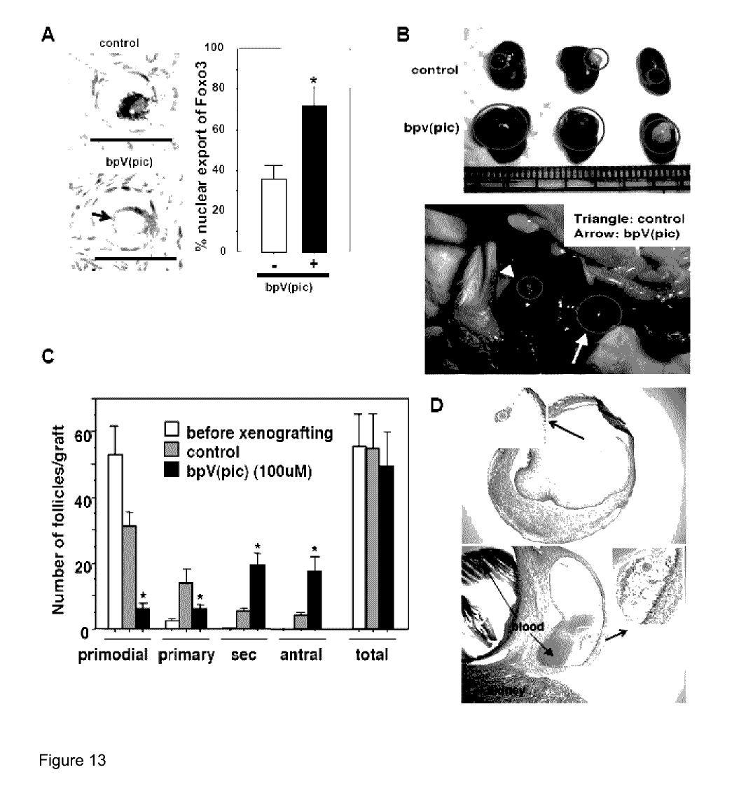

To activate human dormant follicles, ovarian cortical fragments containing primordial follicles were obtained from patients with benign ovarian tumor. After cutting into 1 mm³ cubes, cortical tissues were treated with bpV(pic). As shown in FIG. 13A (left panel), nuclear exclusion of Foxo3 was evident in the treated group as compared with controls at 1 h after culture, showing a 2-fold increase (FIG. 13A, right panel). Pairs of cortical cubes were treated with bpV(pic) or saline for 24 h before xeno-transplantation into each side of the kidney capsule of ovariectomized, SCID (Severe Combined Immunodeficiency) mice. Three days after transplantation, animals were treated i.p. with FSH (1 IU/animal) every 48 h for 24 weeks. At 36 h before sample retrieval, hCG (20 IU/animal) was injected subcutaneously. As shown in FIG. 13B, major increases in graft sizes were apparent in the bpV(pic)-treated group as compared with controls (upper panel: paired grafts with and without bpV(pic) treatment; lower panel: in situ kidney picture of one host). Serial sectioning was performed for four pairs of ovarian grafts together with ovarian cubs before xeno-transplantation. Follicle counting indicated each graft contained ˜50 follicles (FIG. 13C). Before xeno-grafting, 96% of follicles were at the primordial stage with <4% at the primary stage and none at more advanced stages. After transplantation, 40 and 89% of follicles left the dormant primordial pool and developed into larger follicles in control and PTEN inhibitor-treated groups, respectively. In the bpV(pic)-treated group, ˜4-fold increases in the number of secondary and antral follicles were found. Oocyte maturation status of large follicles was also evaluated. In the control group, 8 antral follicles (>1 mm diameter) were present in four ovarian grafts with 7, 0, and 1 oocytes at germinal vesicle (GV), metaphase I (MI), and metaphase II (MII) stages, respectively. In paired grafts treated with bpV(pic), 32 antral follicles were found with 5, 15, and 12 oocytes at GV, MI, and MII stages, respectively. Thus, 4- and 27-fold increases in the number of antral follicles and mature (MI & MII) oocytes were found after bpV(pic) treatment. As shown in FIG. 13D, two representative preovulatory follicles in the bpV(pic)-treated group contained mature MII oocytes (inset).

We performed short-term and ovary-specific treatment of rodent and human ovaries with a PTEN inhibitor and/or a PI3K activator to increase Foxo3 nuclear extrusion in primordial oocytes, leading to the activation of dormant primordial follicles. Subsequent allo- or xeno-transplantation into kidney capsules of FSH-treated hosts allowed optimal follicle development. Once activated, follicles in grafts continue to grow to the antral stage with oocytes capable of undergoing nuclear maturation. For activated murine follicles, mature oocytes could be retrieved for in vitro fertilization and embryo transfer, followed by the delivery of healthy pups. Because epigenetic modification of DNA methylation in the differential methylated regions of key imprinted genes took place in oocytes during folliculogenesis and increased frequencies of imprinting disorders (e.g. Angelman and Beckwith-Wiedemann Syndromes) are associated with assisted reproductive technology for human infertility treatment, we also examined the methylation of two maternally imprinted (Igfr2 and Lit1) and one paternally imprinted (H19) genes in mature oocytes. We found similar patterns for oocytes from activated and super-ovulated control ovaries.

Using in vitro cultures, mutant animals, specific inhibitors, and passive immuno-neutralization tests, several ovarian paracrine factors have been found to be important for the activation of cultured murine primordial follicles, including kit ligand, platelet-derived growth factor, neurotrophins, leukemia inhibitory factor, vascular endothelial growth factor, bone morphogenetic proteins, and FGF proteins. Although the exact factors involved in the activation of few primordial follicles to initiate growth under physiological states are still unknown, it is possible that key tyrosine kinase receptors respond to their ligands in oocytes by direct binding and activation of downstream PI3K and Akt enzymes. In addition, some factors could inhibit PTEN activity, also leading to increases in Akt phosphorylation. Indeed, treatment with the kit ligand activated Akt phosphorylation and suppressed Foxo3 activity in murine oocytes. Binding of tyrosine auto-phosphorylation sites on activated receptors to the SH2 domains of p85, the regulatory subunit of PI3K, releases an autoinhibitory constraint to stimulate the catalytic subunit of PI3K. To stimulate PI3K-Akt-Foxo3 signaling in primordial oocytes, we treated ovaries with a pan-specific PI3K activator. The synthetic 740Y-P peptide has a phosphorylated tyrosine residue with flanking sequences identical to the interaction site of activated PDGF receptor, together with a protein transduction domain (16 residues) of the fly Antennapedia protein to facilitate plasma membrane penetration. 740Y-P is a potent stimulator of PI3K activity and mitogenic responses in myoblast cells and promotes primordial germ cell migration by mimicking the action of the receptor tyrosine kinase c-kit. This PI3K-activating peptide likely increases intracellular PIP3 levels, mimicking the effects of ovarian ligands for tyrosine kinase receptors such as kit ligand, platelet-derived growth factor, leukemia inhibitor factor, and neurotrophins. Coupled with the prevention of PIP3 conversion to PIP2 by the PTEN inhibitor, the PI3K activator stimulated nuclear exclusion of Foxo3 and follicle activation. With subsequent optimal follicle growth in kidney capsules, the present approach allowed efficient activation and development of dormant follicles to derive mature oocytes.

Most primordial follicles are dormant for months or decades in the ovary, likely due to local inhibitory signals. In contrast to the continuous activation of the PI3K-Akt-Foxo3 signaling pathway in PTEN null mice, our findings indicated that murine and human follicles only require short-term treatment to initiate growth. Once activated, follicles continue to grow; they appear to be non-responsive to the inhibitory signals to remain dormant or are producing local stimulating factors to sustain growth. Although the development of preantral follicles are not gonadotropin-dependent, FSH treatment promotes early follicle development. Early studies indicated that 2 and 6 months are required for primordial follicles to reach the preovulatory stage in mouse and human, respectively. After treating hosts with FSH for only 14 days, murine primordial follicles in grafts progressed to the preovulatory stage. For xeno-transplanted human follicles, it is interesting to investigate if a shorter duration is sufficient for antral follicle development. No malignancy was found in xenografted human ovaries or hosts during six months of transplantation, indicating the present short term exposure to the PTEN inhibitor may have minimal adverse effects despite the known function of PTEN as a tumor suppressor gene.

Earlier studies demonstrated the delivery of only few live offspring by IVF of oocytes derived from cryopreserved primordial mouse follicles after in vivo transplantation, likely due to inefficient activation of dormant follicles. Likewise, only few human antral follicles developed from primordial follicles after xeno-transplantation into immune-deficient mice, One case of successful ovarian transplantation between monozygotic twins discordant for premature ovarian failure has been reported and the patient delivered a healthy baby. In addition, ovarian auto-transplantation to the forearm has been successfully performed in women undergoing sterilizing cancer therapy but less than a dozen pregnancies has been reported. Although primordial follicles are more resistant to cryo-damage as compared with larger follicles, ovarian cortical tissue cryopreservation for fertility preservation is still inefficient. The present follicle activation protocol could substantially increase the efficacy to generate mature eggs from patients after auto-transplantation of fresh or frozen ovarian tissues. Although with low efficiency, advances have been made to develop murine primordial in vitro through oocyte maturation and fertilization to allow the birth of live pups. Coupled with the present follicle activation approach, future improvement of follicle culture methods could by-pass the need for in vivo transplantation to generate mature oocytes.

Female mammalian gonads, unlike their male counterparts, have limited number of mature germ cells and the present approach allows the generation of a large number of mature oocytes. Peri-menopausal women and patients suffering from primary ovarian insufficiency/failure have no antral follicles but a limited number of primordial follicles are still present. The present follicle activation approach could benefit infertile patients with a diminishing pool of dormant follicles by providing a large supply of mature female germ cells. The same approach could also allow the generation of mature oocytes from primordial follicles of other mammals, including endangered species and economically important animals.

### Materials and Methods

Experimental Animals:

Mice were obtained from Charles River lab (Wilmington, Mass.) and housed in the animal facility at Stanford University. For xeno-transplantation of human ovarian grafts, female severely compromised immunodeficient (SCID) mice at 4 months of age were obtained from CLEA Japan (Tokyo, Japan) and house at Akita University. Mice were maintained under controlled lighting conditions (12L:12D) with free access to food and water. Animal care was consistent with institutional and NIH guidelines and protocols approved by local committees. Ovaries of three-day-old B6D2F1 females were used for short term incubation and transplantation into kidney capsules of adult female mice of the same strain at 8˜10 weeks of age. For IVF and imprinting tests, 25-day-old B6D2F1 female mice were used to collect ovulated oocytes as positive controls. Pseudo-pregnant CD-1 mice at 8-weeks old were used as recipients for 2-cell embryo transfer.

Short Term In Vitro Incubations:

Paired ovaries were excised, washed three times in L-15 medium containing 3 mg/ml BSA before transferred individually to culture plate inserts (Millipore, Billerica, Mass.). From a given donor, one ovary served as the control and the contra-lateral one was treated with 100 uM bpV(pic) (Calbiochem, La Jolla, Calif.) and/or 500 ug/ml 740Y-P (Tocris, Ellisville, Mich.) for 24 h. After media change, the experimental group was further treated with medium containing only 740Y-P for another 24 h before transplantation. Ovaries were cultured in Waymouth medium MB752/1 supplemented with 0.23 mM pyruvic acid, 50 mg/I streptomycin sulfate, 75 mg/I penicillin G, 3 mg/ml BSA, 10% fetal bovine serum and 0.03 IU/ml FSH (Puregon, NV Organon, Oss, The Netherlands). Media (400 ul) were placed below the membrane insert to allow the covering of ovaries with a thin layer of medium. In some experiments, one ovary from the pair was cultured with bpV(pic) plus 740Y-P whereas the contralateral one was treated with bpV(pic) plus 740Y-P together with the Akt inhibitor SH-5 (50 uM, Calbiochem) or the PI3K inhibitor Wortmannin (25 uM, Calbiochem), added 1 h earlier.

Ovarian Transplantation and Follicle Counting:

Host animals were anesthetized with Ketamine hydrochloride (80 mg/kg, Phoenix, St. Joseph, Mo.) and Xylazine (16 mg/kg, Sigma, St. Louis, Mo.). Kidneys were externalized through a dorso-horizontal incision and paired ovaries (control and treatment groups) from the same donor were randomly inserted under the kidney capsule of the same host that was ovariectomized to increase endogenous gonadotropin levels. One day after transplantation, hosts were treated daily with 2 IU FSH and some animals were sacrificed at 14 days after transplantation to assess follicular development. Grafted ovaries were collected and fixed in 10% buffered formalin for 12 h, embedded in paraffin, serially sectioned at a thickness of 4 um, and then stained with hematoxylin and eosin. Follicle dynamics was evaluated as previously described. Follicles were only counted when the dark-staining nucleolus was seen within the nucleus of the oocytes to prevent recounting of the same follicle. Sections were counted by two independent individuals for comparison. To assess oocyte maturation, some animals were given a single i.p. injection of hCG (10 IU) at 18 days after transplantation. Grafted ovarian tissues were collected 8 h after hCG injection before serial sectioning and staining to assess nuclear maturation (germinal vesicle breakdown) of oocytes in antral follicles.

Immunohistochemistry and BrdU Labeling:

Ovaries from day 3 mice were incubated with bpV(pic) and 740Y-P. Ovaries treated with bpV(pic) and 740Y-P for 6 h were fixed in 10% buffered formalin for 12 h and used to detect the expression of Foxo3. Some ovaries were treated for 48 h before fixing in Bouin's buffer and used for AMH, a marker for growing follicles. After embedding and sectioning at 5 μm, samples were deparaffinized, rehydrated, and endogenous peroxidase activity was blocked by incubation in 1.5% peroxide in methanol for 20 min. Antigen retrieval pretreatment was carried out by boiling the sections in 0.01 M citrate buffer, pH 6.0, for 10 min and cooling slowly. Immunohistochemical analyses were performed by the Histostain kit (Invitrogen, Camarillo, Calif.) using antibodies to Foxo3 (Cell signaling, Danvers, Mass.) and AMH (Santa Cruz Biotechnology, Santa Cruz, Calif.) overnight at 4 C. For some sections, primary antibodies were replaced with non-immune rabbit IgG as negative controls. For evaluating cell proliferation, hosts at five days after transplantation were treated with a single i.p. injection of BrdU (Invitrogen) at 1 ml/100 g BW before collecting the grafts at 12 h later for the analyses of BrdU labeling using a BrdU staining Kit (Invitrogen).

DNA Methylation Analysis:

At 18 days after transplantation, hosts received a single injection of hCG (10 IU) for 14 h. Mature oocytes (n=30) were collected from ovaries to analyze epigenetic changes. For control samples, mature oocytes (n=30) were collected from oviducts of super-ovulated immature mice treated with PMSG (48 h), followed by 10 IU of hCG to induce ovulation. Genomic DNA was prepared using the PicoPure DNA extraction kit (Arcturus, Sunnyvale, Calif.) and treated with bisulfate according to the protocol of the EpiTect bisulfite Kit (Qiagen, Valencia, Calif.). The bisulfate-converted DNA was PCR amplified by using nested primers and the EpiTect MSP PCR kit (Qiagen, Valencia, Calif.) for Igf2r, Lit1, and H19 genes. PCR products were purified and cloned into a pGEM T-Easy Vector (Promega) and 15-20 samples from each gene were sequenced using an ABI PRISM 3100 Genetic Analyzer (Applied Biosystems, Foster City, Calif.) to estimate methylation patterns.

IVF and Embryo Transfer:

At 18 days after transplantation, hosts were treated with 10 IU hCG and grafted ovaries were collected at 14 h later into the M2 medium (Millipore, Phillipsburg, N.J.). Ovaries were then punctured to release oocytes in media containing 0.3 mg/ml hyaluronidase (Sigma). Retrieved oocytes were scored for maturity and only metaphase I and 11 oocytes were used for in vitro fertilization. As controls, B6D2F1 mice at 25 days of age received one i.p. injection of PMSG followed, at 48 h later, with another injection of hCG. At 14 h later, MI and MII oocytes were collected from the oviductal ampula. To obtain donor sperm, sperm were collected from B6D2F1 mice (10 to 14 weeks of age) into the human tubal fluid media (Millipore, Phillipsburg, N.J.) and incubated under oil for 1 h at 37 C in a 5% CO2 and 95% air. Oocytes were then placed in 250 ul media with sperm (2-3×10⁵/ml). Fertilization was carried out for 6 h and inseminated oocytes were removed into 20 ul droplets of KSOM media (Millipore) under mineral oil and incubated at 37 C. Two-cell embryos were scored 24 h later and all oocytes not inseminated were removed from the droplet. Some embryos were allowed to develop to the blastocyst stage as previously described. For embryo transfer, 2-cell embryos were transferred into the oviducts of pseudopregnant CD1 mice mated with vasectomized males of the same strain.

Human Follicle Activation:

Ovarian fragments were obtained from patients with benign ovarian tumor but exhibiting normal menstrual cycles. Informed consent from the patient and approval from the Human Subject Committee of Akita University were obtained. Ovarian cortex was isolated and cut into small cubes (˜1 mm³). After treatment of cortical cubes for 1 h with bpV(pic), some samples were used for immunohistochemical staining for Foxo3. Some ovarian cubes were also cultured in MEM-a medium containing 10% human serum albumin (Mitsubishi Tanabe Pharma Corp, Osaka, Japan), 1% antibiotic/antimycotic solution (Invitrogen), and 0.3 IU/ml FSH. Pairs of cortical cubes were treated with bpV(pic) (100 mM) or saline for 24 h before xeno-transplantation into each side of the kidney capsule of ovariectomized, immune-deficient SCID mice. An i.p. injection of antibiotic (4 μg/g body weight; gentamicin; Invitrogen) was given while animals were anesthetized by 2,2,2-tribromoethanol (0.4 mg/g body weight; avertin; Sigma). Three day after transplantation, animals were treated i.p. with FSH (1 IU/animal) every 48 h for 24 weeks. At 36 h before sample retrieval, hCG (20 IU/animal) was injected subcutaneously. Ovarian grafts were recovered for histological analyses and follicle counting.

Statistical Analysis:

Mann-Whitney U test and one-way ANOVA were used to evaluate differences between groups. Data are mean±SEM.

All publications and patent applications cited in this specification are herein incorporated by reference as if each individual publication or patent application were specifically and individually indicated to be incorporated by reference. The publications discussed herein are provided solely for their disclosure prior to the filing date of the present application. Nothing herein is to be construed as an admission that the invention is not entitled to antedate such a disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-17. (canceled)

18. A method of activating dormant mammalian ovarian primordial follicles, the method comprising:
contacting at least one dormant mammalian ovarian follicle with: (a) an inhibitor of PTEN for a period of at least 1 hour but not more than 3 days, and (b) an activator of PI3K,
in doses effective to activate the at least one dormant mammalian ovarian follicle, thereby generating an active follicle.

19. The method of claim 18, wherein the at least one dormant mammalian ovarian follicle is a human follicle.

20. The method of claim 18, wherein said contacting with an inhibitor of PTEN is for a period of at least 1 hour but not more than 2 days

21. The method of claim 18, wherein the activator of PI3K is 740Y-P.

22. The method of claim 18, wherein said inhibitor of PTEN is an oxovanadium compound.

23. The method of claim 18, wherein said inhibitor of PTEN is bisperoxo(picolinato) oxovanadate (V).

24. The method of claim 18, wherein the contacting with (a) and with (b) is performed in vitro.

25. The method of claim 24, wherein the activated follicle is transplanted into an adult recipient.

26. The method of claim 25, wherein the recipient is autologous to the activated follicle.

27. The method of claim 18, wherein the contacting with (a) and with (b) is performed in vivo.

28. A method of activating dormant mammalian ovarian primordial follicles, the method comprising:
contacting at least one dormant mammalian ovarian follicle with (a) an inhibitor of PTEN for a period of at least 1 hour but not more than 3 days, and (b) an activator of PI3K that binds to PI3K,
in doses effective to activate the at least one dormant mammalian ovarian follicle, thereby generating an active follicle.

29. The method of claim 28, wherein the at least one dormant mammalian ovarian follicle is a human follicle.

30. The method of claim 28, wherein said contacting with an inhibitor of PTEN is for a period of at least 1 hour but not more than 2 days

31. The method of claim 28, wherein the activator of PI3K is 740Y-P.

32. The method of claim 28, wherein said inhibitor of PTEN is an oxovanadium compound.

33. The method of claim 28, wherein said inhibitor of PTEN is bisperoxo(picolinato) oxovanadate (V).

34. The method of claim 28, wherein the contacting with (a) and with (b) is performed in vitro.

35. The method of claim 34, wherein the activated follicle is transplanted into an adult recipient.

36. The method of claim 35, wherein the recipient is autologous to the activated follicle.

37. The method of claim 28, wherein the contacting with (a) and with (b) is performed in vivo.
